(19) United States
(12) Patent Application Publication
(10) Pub. No.: US 2025/0366767 A1
(43) Pub. Date: Dec. 4, 2025

(54) SURFACE FLEXIBLE ELECTRODE FOR CENTRAL NERVOUS SYSTEM AND METHOD FOR PREPARING SAID ELECTRODE

(71) Applicant: SHANGHAI STAIRMED TECHNOLOGY CO., LTD., Shanghai (CN)

(72) Inventors: Xue LI, Shanghai (CN); Zhengtuo ZHAO, Shanghai (CN); Jianing YANG, Shanghai (CN)

(21) Appl. No.: 18/875,626

(22) PCT Filed: Jun. 29, 2022
(86) PCT No.: PCT/CN2022/102371

§ 371 Date: Dec. 16, 2024

(30) Foreign Application Priority Data
Jun. 17, 2022 (CN) 202210689965.0

(51) Int. Cl. A61B 5/37; A61B 5/263; A61B 5/293; A61N 1/05; G03F 7/00

(52) U.S. Cl. CPC A61B 5/37; A61B 5/263; A61B 5/293; A61N 1/0531; G03F 7/0015; A61B 2562/125

(57) ABSTRACT

The present disclosure provides a surface electrode for a central nervous system and a method for preparing said electrode. The surface electrode includes: at least one implantable and flexible electrode plate, wherein each of the at least one electrode plate includes: a wire, located between a first insulating layer and a second insulating layer of the flexible electrode; and an electrode site, located on the outer surface of at least one of the first insulating layer and the second insulating layer, and electrically coupled to the wire by means of a through hole in the at least one insulating layer. The surface electrode is configured to be flattened and attached to the surface of the central nervous system biological tissue after implantation.

### TECHNICAL FIELD

The present disclosure relates to the technical field of life science, and more specifically to an ultra-thin and ultra-flexible surface flexible electrode for a central nervous system and a method for preparing said electrode.

### BACKGROUND

Cerebrum and spinal cord are centers of a human nervous system, wherein the cerebrum affects various functions of human survival and life, such as speech, movement and memory, and the spinal cord is responsible for transmission of human body control, and closely related to transmission of body control and sensory signals under the cerebrum. At present, in medical or research occasions where a neuronal mechanism for regulation of human cerebrum and limbs is explored, such as treatment of organic lesions of the cerebrum and spinal cord, enabling a paralyzed or limb-deficient patient to independently interact with the outside, positioning, recording and functional electrical stimulation of distribution of surface neuronal electrical signals of the central nervous system are required. By use of a surface flexible electrode technology for the central nervous system, quick, accurate and minimally invasive recording and functional positioning, electrical stimulation of distribution of invasive neuron electrical signals are expected.

At present, the existing surface electrode for the central nervous system is designed by: manufacturing a polyimide substrate by using a tape casting method, and manufacturing an inner wire (by referring to manufacturing of an FPC circuit board) by using a chemical deposition method, the inner wire usually containing nickel copper, and an electrode contact site usually containing nickel gold; manufacturing the electrode by using a micro-nano processing technology, a material of the electrode substrate being PI or PDMS and a material of the inner wire being usually gold. However, due to the material and electrode structure selected in the above manufacturing methods, defects in hardness or thickness or the like will occur on the surface electrode, and ideal signal recording and stimulation cannot be achieved in terms of shape and channel number.

### SUMMARY

The present application provides an ultra-thin and ultra-flexible surface electrode for a central nervous system and a method for preparing the electrode.

According to a first aspect of embodiments of the present disclosure, an ultra-thin and ultra-flexible surface electrode for a central nervous system and a method for preparing the electrode are provided. The surface electrode comprises: at least one implantable and flexible electrode sheet, wherein each electrode sheet comprises: a wire located between a first insulating layer and a second insulating layer of the flexible electrode; and an electrode site located on an outer surface of at least one insulating layer of the first insulating layer or the second insulating layer and electrically coupled to the wire through a via hole in the at least one insulating layer, wherein, the wire has a width of 10 nm to 500 μm, and the surface electrode is configured to be flattened and fit against a tissue surface of the central nervous system after implantation into a cerebrum.

According to a second aspect of the embodiments of the present disclosure, there is provided a method for manufacturing an ultra-thin and ultra-flexible central nervous system surface electrode, the electrode comprising the surface electrode according to the first aspect, the method comprising: manufacturing, layer by layer, the first insulating layer, a wire layer, the second insulating layer and an electrode site layer, wherein, before manufacturing the electrode site layer, the via hole is manufactured at a position in the second insulating layer that corresponds to the electrode site by a patterning method.

An advantage of the embodiments according to the present disclosure is that, the central nervous system surface electrode involved in the present application can be used for both electrical signal acquisition of biological neural tissue (e.g., cerebral cortex or spinal cord surface) and functional electrical stimulation to the biological neural tissue (e.g., cerebral cortex or spinal cord surface). By thinning the electrode, its bending rigidity can be reduced, thereby improving mechanical performance mismatch between the electrode and the tissue, and finally, a long-term stable electric signal recording and stimulation interface is provided.

Another advantage of the embodiments according to the present disclosure is that, the central nervous system surface electrode disclosed in the present application can be shaped as needed, thereby making the electrode array applicable to different central nervous regions or other model animals. In addition, the electrode can be designed into a different number of layers, a different number of contact points, different sizes and contact point distributions according to different requirements, which is of important significance in neuroscience research and rehabilitation medical application.

It should be appreciated that, the above advantages need not all be concentrated in one or some specific embodiments, but may be partially dispersed among different embodiments of the present disclosure. The embodiments according to the present disclosure may have one or some of the above advantages, or may alternatively or additionally have other advantages.

By the following detailed description of exemplary embodiments of the present application with reference to the accompanying drawings, other features of the present application and advantages thereof will become more apparent.

### BRIEF DESCRIPTION OF THE DRAWINGS

FIG. 1 is a schematic view illustrating at least part of a surface electrode according to an embodiment of the present disclosure.

FIG. 2 is an exploded view illustrating at least part of a surface electrode according to an embodiment of the present disclosure.

FIG. 3 is a schematic view illustrating a support bracket for an ECOG electrode according to an embodiment of the present disclosure.

FIG. 4 is a sectional view illustrating a support bracket for an ECOG electrode according to an embodiment of the present disclosure.

FIG. 5 is a schematic view illustrating implantation of an ECOG electrode via a support bracket according to an embodiment of the present disclosure.

FIG. 6 is another schematic view illustrating a support bracket for an ECOG electrode according to an embodiment of the present disclosure.

FIG. 7 is another schematic view illustrating implantation of an ECOG electrode via a support bracket according to an embodiment of the present disclosure.

FIG. 8 is yet another schematic view illustrating a support bracket for an ECOG electrode according to an embodiment of the present disclosure.

FIG. 9 is yet another schematic view illustrating implantation of an ECOG electrode via a support bracket according to an embodiment of the present disclosure.

FIG. 10 is a flow diagram illustrating a method for manufacturing an ECOG electrode according to an embodiment of the present disclosure.

FIG. 11 is a schematic view illustrating a method for manufacturing an ECOG electrode according to an embodiment of the present disclosure.

### DETAILED DESCRIPTION

Various exemplary embodiments of the present disclosure will be described in detail below with reference to the accompanying drawings. It should be noted that: relative arrangements of components and steps, numerical expressions and numerical values set forth in these embodiments do not limit the scope of the present disclosure unless specifically stated otherwise.

The following description of at least one exemplary embodiment is merely illustrative in nature and is in no way intended to limit this disclosure, its application or use. That is, the structures and methods herein are shown in exemplary ways to illustrate different embodiments of the structures and methods in the present disclosure. Those skilled in the art will understand, however, that they of are merely illustrative exemplary ways in which the disclosure may be implemented, rather than exhaustive ways. Furthermore, the drawings do not need to be drawn to scale, and some features may be enlarged to show details of specific components.

Techniques, methods, and devices known to one of ordinary skill in the related art may not be discussed in detail but are intended to be part of the granted specification where appropriate.

There already have design and use of the central nervous system surface electrode in the prior art, but technical problems to be solved exist in practical applications. It should be noted that, in the following embodiments of the present application, an electrocorticogram (ECOG electrode for short below) for a cerebrum surface will be taken as an example. Specifically, due to the used material, manufacturing method, hierarchical structure, etc., the ECOG electrode is excessively thick or hard, with a clear damage (such as squeeze or scratch) to the cerebrum and insufficient adhesion to the cerebrum surface. During long-term use, friction between a hard tube and a nerve tract will cause damage to the nerve, a scar formed on the nerve will affect recording and stimulation of signals, and may also cause a serious long-term immune response after implantation, resulting in incapability of performing long-term stable signal recording and stimulation by the electrode. A conventional ECOG electrode cannot be compatible with a CT/MRI experiment, and most of existing commercial ECOG electrodes cannot be customized in shape and are usually rectangular, so that they cannot be implanted into a narrow or deep cerebrum area and cannot fully cover the cerebrum area having an extremely large area; due to large electrode sites and small number of channels, high-flux high-precision (such as 128-channel) signal recording and stimulation once cannot be implemented.

In order to solve the above technical problems, in the present application, an ultra-flexible material and design is adopted to replace the traditional ECOG electrode, and polymer is used as an insulating layer to wrap a conductive material, and by reducing a thickness of the electrode, its bending rigidity can be reduced, thereby improving mechanical performance mismatch between the electrode and tissue, and finally, a long-term stable electric signal recording and stimulation interface is provided. The use of non-magnetic metal and ultra-thin design allows the electrode to be compatible with the CT/MRI, and the ultra-thin design and an auxiliary implantation device enable the electrode to go deep into the narrow or deep cerebrum area. Electrode site arrangement is optimized by using a micro-nano technology, making the electric signal acquisition in high flux.

In summary, the technical solution of the present disclosure mainly relates to a flexible electrode for recording and stimulation of cerebral cortex electrophysiological signals, which is characterized by having a mesh screen and a non-regularity structure, so that targeted coverage can be performed on a required cerebral cortex, with technical effects of high coverage, high fit and low trauma. The electrode is flexible in design, so that the number of its structural layers, the number of channels, its shape and size, site distribution, compatibility with a backend device interface and the like can be changed according to different product requirements. The electrode implantation adopts a minimally invasive solution, so that an inflammatory reaction of the cerebral tissue after the implantation surgery and during electrode retention can be reduced. Meanwhile, whether the electrode is compatible with a medical detection method such as the MRI/CT is optional, and it can also be integrated with a chip to implement an integrated system of electrode and chip. Since the ECOG electrode in the present disclosure can simultaneously perform signal acquisition and stimulation and different lead channels are independent of each other, it is possible to perform simultaneous or non-simultaneous leading-in stimulus and signal recording experiments in same or different cerebrum areas, and it is also possible to lead in, in different channels, different stimuli, which are associated with behaviors, and to explore the influence of a certain stimulus, certain simultaneous non-same-site stimuli, and sequential stimuli on behaviors of subjects.

The technical solution of the present application will be described in detail below in conjunction with the accompanying drawings.

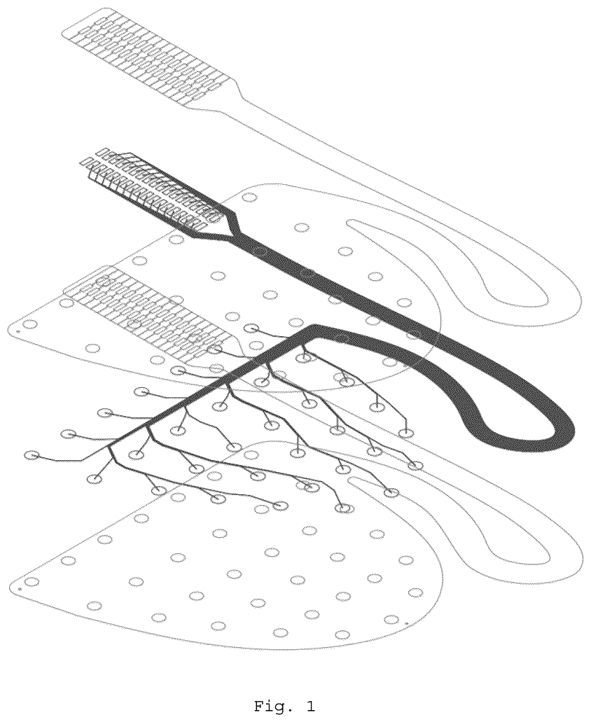

FIG. 1 illustrates a schematic view of a surface electrode for a central nervous system. In one embodiment according to the present disclosure, an ECOG electrode for a cerebrum surface is taken as an example of the surface electrode. The electrode is used for performing targeted coverage on different cerebrum areas, which facilitates detection of electric signal activity of a specific cerebrum area when the subject's limb and cerebrum behaviors act or sense, so that compared with a common strip electrode, the electrode is mostly in a form of a sheet, which can provide wide coverage for a half/whole cerebrum, and high coverage of the half/whole cerebrum facilitates differential detection of electric signal activity of different cerebrum areas when the subject's specific limb and cerebrum behaviors act, and a time sequential relation of the electric signal activity of different cerebrum areas in a certain period of time. FIG. 1 schematically shows that the ECOG electrode includes at least three layers, which respectively are: a top insulating layer that leaves an electrode site exposed, an intermediate layer including a wire and the electrode site, and a bottom insulating layer. The ECOG electrode is implanted into the cerebrum area by means of surgery and can be kept flattened and fit against a cerebral cortex.

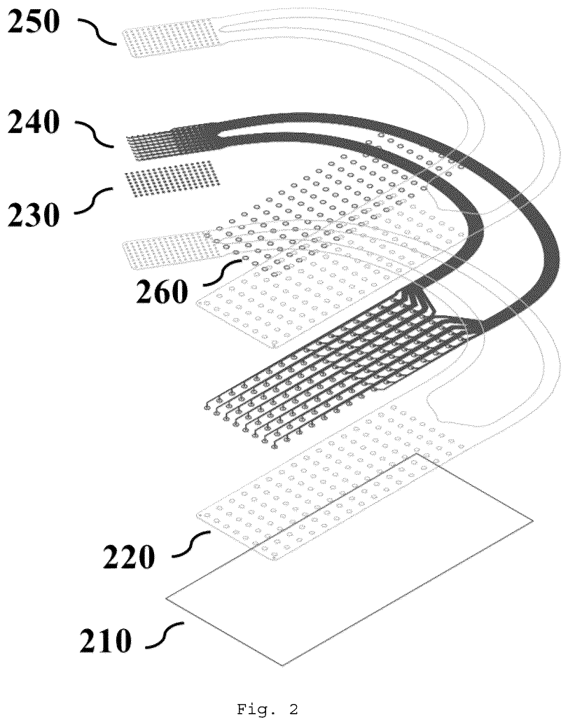

FIG. 2 is an exploded view illustrating at least part of a flexible electrode according to an embodiment of the present disclosure. Note that FIG. 2 is merely illustrative, where the relative sizes and design shapes of various layers are not necessarily as shown in FIG. 2, and in practice a flexible electrode for ECOG includes, for example, at least one implantable and flexible electrode sheet, wherein a size of an electrode site area may cover a larger cerebrum area. The electrode is mainly manufactured by a micro-nano processing technology, and a multilayer structured electrode with a thickness of nanometers can be manufactured, with high yield and stable quality. Specifically, as shown in FIG. 2, the electrode specifically includes a flexible separation layer 210, a first insulating layer 220, a connection layer 230 with a circuit board, a wire layer 240, a second insulating layer 250, an electrode site layer 260, and the like. It should be understood that the layers of the flexible electrode shown in FIGS. 1 and 2 are merely non-limiting examples, and for the ECOG electrode in the present disclosure, one or more of the layers may be omitted, and more other layers may be included.

As shown in FIG. 1, the wire in the electrode comprises a plurality of wires located in the wire layer and spaced apart from each other, wherein, the electrode site in the electrode comprises a plurality of electrode sites each electrically coupled with one of the plurality of wires through a corresponding via hole in the bottom insulating layer. The electrode has good flexibility so that it can be partially or fully implanted into biological tissue to acquire or apply electrical signals from or to the biological tissue. The wire layer of the electrode shown in FIG. 1 includes a plurality of wires, however, it should be understood that the electrode in the present disclosure may include a single wire or other specified number of wires in different embodiments. These wires may have a width and thickness of nanometers or micrometers, as well as a length in several orders of magnitude (such as centimeters) greater than the width and thickness as needed. In the embodiment according to the present disclosure, the shapes, sizes, and the like of these wires are not limited to the ranges listed above, but may be changed according to design requirements.

Specifically, the electrode may include a first insulating layer 220 located at the bottom of the electrode and a second insulating layer 250 located at the top of the electrode. The insulating layer in the electrode may refer to an outer surface layer in the electrode that functions as insulation. Since the insulating layer of the flexible electrode needs to be in contact with the biological tissue after implantation, it is required that a material of the insulating layer has good biocompatibility while having good insulation. In the embodiment of the present disclosure, the material of the insulating layers 220, 250 may include polyimide (PI), polydimethylsiloxane (PDMS), parylene, epoxy, polyamide-imide (PAI), and the like. In addition, the insulating layers 220, 250 are also a major part in the multi-channel mesh electrode that provides strength. An excessively thin insulating layer will reduce the strength of the electrode, an excessively thick insulating layer will reduce the flexibility of the electrode, and implantation of the electrode including the excessively thick insulating layer will bring great damage to the organism. In the embodiment according to the present disclosure, the insulating layers 220, 250 may have a thickness of 100 nm to 300 μm.

The wire layer in the electrode are distributed in the wire layer 240 between the first insulating layer 220 and the second insulating layer 250. In the embodiment according to the present disclosure, each electrode sheet may include one or more wires located in the same wire layer 240. For example, it can be clearly seen from FIG. 2 that, the wire layer 240 includes a plurality of wires, wherein each wire includes an elongated body and an end corresponding to a corresponding electrode site. The wire may have a width of, for example, 10 nm to 500 μm, and an interval between the wires may be, for example, as little as 10 nm. It should be understood that the shape, size, interval, etc. of the wire are not limited to the ranges listed above, but may be changed according to design requirements.

In the embodiment according to the present disclosure, the wires in the wire layer 240 may be a film structure including a plurality of layers stacked in a thickness direction. A material of these layers may be a material that may enhance adhesion, ductility, conductivity and the like of the wire. As a non-limiting example, the wire layer 240 may include a conductive layer and an adhesion layer stacked, wherein a material of the adhesion layer in contact with the insulating layer 220 and/or 250 is a metallic or non-metallic adhesion material such as titanium (Ti), titanium nitride (TiN), chromium (Cr), tantalum (Ta), tantalum nitride (TaN), etc., and a material of the conductive layer is a material having good conductivity such as aurum (Au), platinum (Pt), iridium (Ir), tungsten (W), magnesium (Mg), molybdenum (Mo), platinum-iridium alloy, titanium alloy, graphite, carbon nanotube, PEDOT, etc. It should be understood that, the wire layer may also be made of other metallic or non-metallic materials with conductivity, or made of a polymer conductive material and a composite conductive material. In one non-limiting embodiment, the conductive layer of the wires may have a thickness of, for example, 5 nm to 200 μm, and the adhesion layer has a thickness of 1 to 50 nm.

The electrode according to the present disclosure may also include electrode sites located in the electrode site layer 260 above the first insulating layer 220, which may be in contact with biological tissue to directly acquire or apply electrical signals after the implantation into the flexible electrode. The electrode site in the electrode site layer 260 may be electrically coupled to a corresponding wire through a via hole at a position in the first insulating layer 220 that corresponds to the electrode site. When the electrode includes a plurality of wires, the electrode may correspondingly include a plurality of electrode sites in the electrode site layer 260, and the electrode sites are each electrically coupled with one of the plurality of wires by means of a corresponding via hole in the first insulating layer 220.

In one non-limiting embodiment, each electrode site may have a corresponding wire in the wire layer 240. Each electrode site may have a planar size of micrometers and a thickness of nanometers. In the embodiment according to the present disclosure, the electrode site may include a site having a diameter of 1 μm to 500 μm, and an interval between the electrode sites may be, for example, 10 μm to 10 mm. In the embodiment according to the present disclosure, the electrode site may take a shape of a circle, ellipse, rectangle, rounded rectangle, chamfered rectangle, and the like. It should be appreciated that the shape, size, interval, etc. of the electrode site may be selected according to the region of the biological tissue to be recorded.

In the embodiment according to the present disclosure, the electrode site in the electrode site layer 260 may be a film structure including a plurality of layers stacked in a thickness direction. A material of a layer in the plurality of layers that is close to the wire layer 240 may be a material that may enhance adhesion between the electrode site and the wire. As a non-limiting example, the electrode site layer 260 may be a metal film including two stacked layers, wherein, a first layer close to the wire layer 240 is Ti, TiN, Cr, Ta and TaN, and a second layer of the electrode site layer 260 that is exposed is Au. It should be understood that, similarly to the wire layer, the electrode site layer may also be made of other metallic or non-metallic materials with conductivity, such as Pt, Ir, W, Mg, Mo, platinum iridium alloy, titanium alloy, graphite, carbon nanotube, PEDOT, etc.

In the embodiment according to the present disclosure, an exposed surface of the electrode site that is in contact with the biological tissue may also have a surface treatment layer to improve electrochemical properties of the electrode site. A material of the surface treatment layer is any one or a combination of PEDOT, iridium dioxide, porous gold, platinum black (Pt black). As a non-limiting example, the surface treatment layer may be obtained by using an electropolymerization coating of PEDOT: PSS, sputtering an iridium oxide film, growing or sputtering a Pt black film, preparing sponge-like (porous) gold, and the like, and be used for decreasing impedance (such as electrochemical impedance at an operating frequency of 1 kHz) in the case of acquiring electric signals by the flexible electrode, and enhancing a charge injection capability in the case of applying electric signal stimuli by the flexible electrode, thereby improving interaction efficiency.

In the embodiment according to the present disclosure, the electrode may further comprise a flexible separation layer 210. The flexible separation layer 210 in FIG. 2 is mainly used in a manufacturing process of the multi-channel mesh electrode, for which a material is a metallic or non-metallic material such as nickel, chromium, and aluminum, with a property of being specifically removed by a specific substance (such as a solution), so as to separate two parts of the flexible electrode above and below the flexible separation layer, while avoiding damage to the flexible electrode. Specifically, the flexible separation layer can be used for separating the entire electrode or only the flexible part of the electrode from the substrate, separating a flexible substrate from a hard substrate, separating a part where an adhesion force is excessively strong but separation is required, etc. The flexible separation layer 210 is further provided with an adhesion layer, for which a material comprises titanium, titanium nitride, chromium, tantalum or tantalum nitride.

Next, minimally invasive implantation of the ECOG electrode according to the present disclosure will be described. Generally, in the implantation, a support bracket having a micromechanical structure is used, which is capable of delivering the electrode along a gap between central nervous tissue and a bone to a position which is not easily accessible by a conventional surgical operation, such as delivering the electrode through a gap between cerebrum and skull into frontal lobe, etc., which not only can avoid craniotomy and other operations, but also can keep the electrode flattened and unfolded after delivered into the cerebrum, with low trauma to the cerebrum. In other words, in the implantation of the ECOG electrode by using the support bracket (such as implantation of the electrode along a gap between the cerebrum and skull), craniotomy may be avoided to protect a part in the cerebrum area that is prone to trauma, including anterior frontal lobe, posterior occipital lobe, inferior temporal lobe, or central cerebral great vessel. Alternatively, for an electrode implanted to a spine surface, the electrode may, by using a support bracket without or with only partial removal of a backbone, be implanted into a part with a little gap, difficulty in access or easy in a large trauma in a spinal cord, including spinal cord intumescentia, spinal ganglion or spinal artery passage and anastomosis, etc.

A material of the support bracket for the implantion of the ECOG electrode includes, but is not limited to, metal and alloy such as tungsten, platinum, titanium, and magnesium, a polymer material such as polyimide, Polydimethylsiloxane (PDMS), hydrogel, epoxy, and polyethylene, and an inorganic or organic material such as chitosan and polyethylene glycol (PEG), which can be electrolyzed, hydrolyzed, pyrolyzed, and biodegraded. Therefore, the support bracket and/or decomposition products thereof do not generate toxicity to the organism, which can avoid damage to a surgical region where the electrode is implanted.

Generally, the mechanical structure of the support bracket for the implantation includes, but is not limited to, a cantilever, a latch, a linkage mechanism, microfluidics, etc., which allows that when implanted, the electrode is kept in a state of, including, but not limited to, flattened, rolled, wrapped, etc., after implanted and when retained in the cerebrum, it is flattened and fits into the cerebral cortex, and it can be removed from the cerebrum in a state of, including, but not limited to, flattened, rolled, wrapped, etc.

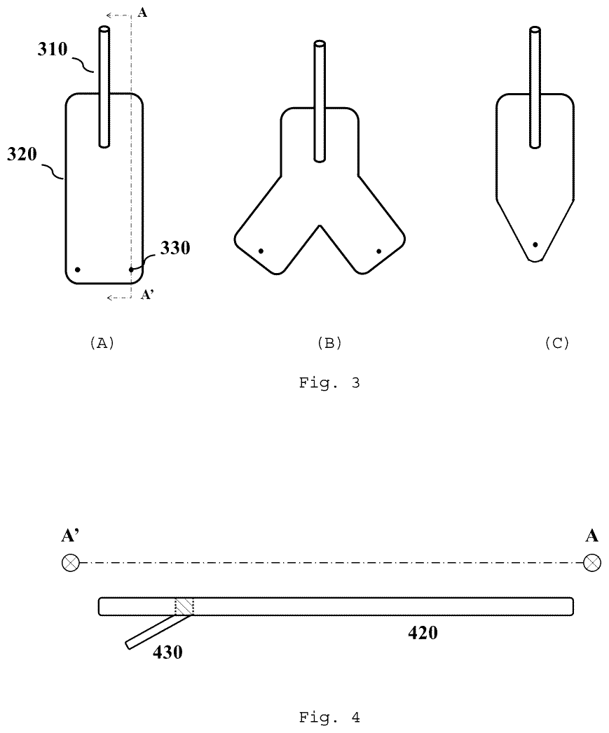

FIGS. 3 to 5 show a non-limiting embodiment of a support bracket for an ECOG electrode. FIG. 3 shows a number of different shapes of a support bracket, which mainly comprises, in an example of FIG. 3(A), a hard handle 310, a support bracket body 320 and an electrode hook 330. A size of the support bracket depends on a specific size and shape angle of the implanted electrode, and a placement angle of the support bracket with respect to the cerebrum area is flexibly defined to reduce the implantation difficulty. The support bracket body 320 has a certain flexibility, so that it is bendable to some extent to adapt to an uneven surgical region in surgical implantation, and has various forms such as a rectangle-like shape in FIG. 3(A) or a Y-shape in FIG. 3(B), or has a small and round head as shown in FIG. 3(C) to prevent scratching the cerebrum surface. The electrode hook 330 is used for hooking a small hole (about 50 μm to 1 mm in size) on the electrode to be implanted, which can be made of metal such as tungsten wire or degradable polymer such as PI and polylactic acid, and can be one or more in number. A section A-A′ of the electrode hook 330 in FIG. 3(A) is shown in FIG. 4, where 420 is the support bracket body, 430 is the electrode hook, and a shadow corresponds to an electrode hook positioning point in FIG. 3, a front of the electrode hook 430 not exceeding a foremost end of the support bracket body 420. It should be noted that a relationship between the size and the shape in FIG. 4 is schematic, and in practical applications, support brackets in different sizes and shapes can be designed according to requirements.

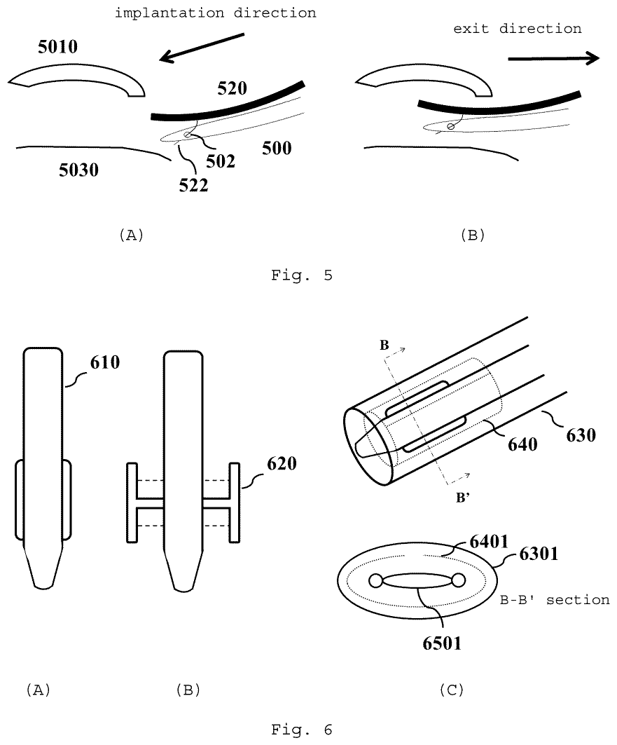

FIG. 5 shows a schematic view of a step of implanting an ECOG electrode via a support bracket according to the foregoing embodiment. As shown in FIG. 5(A), a support bracket 520 carries a flexible electrode 500 for implantation into an cerebrum area, specifically, an electrode hook 522 on the support bracket 520 passes through a small hole 502 on the electrode 500, such that the electrode 500 enters between a skull 5010 and a cerebrum surface 5030 in an implantation direction indicated by an arrow in FIG. 5(A) in a flattened state. Subsequently, as shown in FIG. 5(B), when the electrode 500 reaches a designated position on the cerebrum surface 5030, the support bracket 502 is taken out in an exit direction indicated by an arrow in FIG. 5(B), thereby completing the implantation of the ECOG electrode.

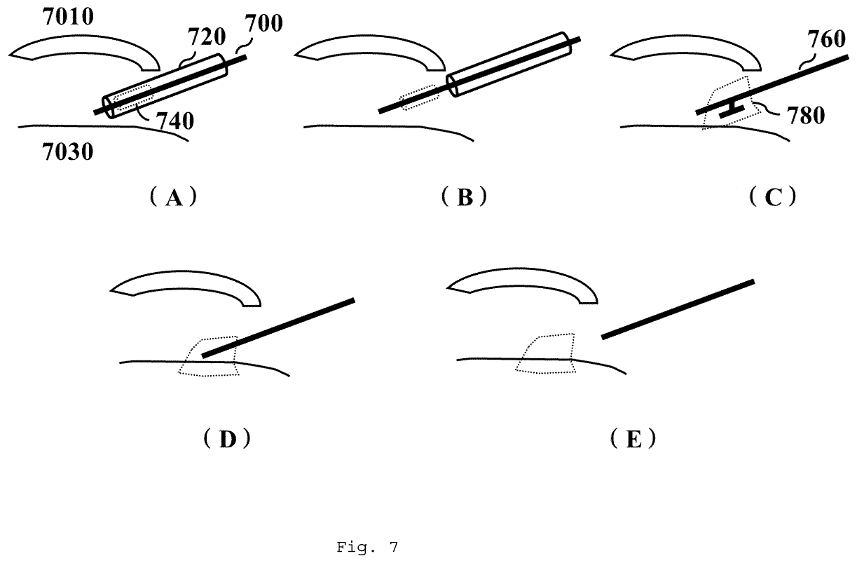

FIGS. 6 to 7 show another non-limiting embodiment of a support bracket for an ECOG electrode. FIGS. 6(a) and 6(B) show a shape of the support bracket, including a flat cylindrical support bracket body 610 and a flattening shelf 620 that can be unfolded. In particular, the flattening shelf 620 may be converted from a folded state of FIG. 6(A) to an unfolded state of FIG. 6(B) by a mechanical device such as pneumatic pressure, a linkage, or other implementations. FIG. 6(C) shows a form of an ECOG electrode ready for implantation, wherein an upper half of FIG. 6(C) is a simplified perspective view of an electrode mounted on a support bracket, and a lower half is a schematic view of a B-B′ section of the perspective view. It should be noted that FIG. 6(C) simplifies the form of the electrode into a relatively standard flat cylinder, and in fact, the fit between the electrode and the support bracket may be designed into any shape as needed, such as a part at a front end that is approximate to a frustum cone. As shown in the figure, 630 is a tubule for protecting the electrode, and 640 is the ECOG electrode wrapped on the support bracket, embodied, on the B-B′ section, as a tubule 6301 wrapped outwards and the ECOG electrode 6401 wrapped around the support bracket 6501 in a rolled state, respectively.

FIG. 7 is a schematic view of a step of implanting an ECOG electrode via a support bracket according to the foregoing embodiment. As shown in FIG. 7(A), a support bracket 700, which has thereon wrapped an electrode 740, is implanted between skull 7010 and cerebrum surface 7030 under protection of a tubule 720. After the support bracket 700, with the electrode 740 carried, reaches a designated position, the tubule 720 is drawn out as shown in FIG. 7(B). Then, as shown in FIG. 7(C), the support bracket 700 is opened into a support bracket 760 with a form of a flattening shelf, and the ECOG electrode is flattened from a rolled state to an electrode 780 by means of the flattening shelf. Next, as shown in FIG. 7(D), the support bracket is restored to the 700 form by folding up the flattening shelf. Finally, as shown in FIG. 7(E), the support bracket 700 is drawn out to complete the flattening and implantation of the electrode.

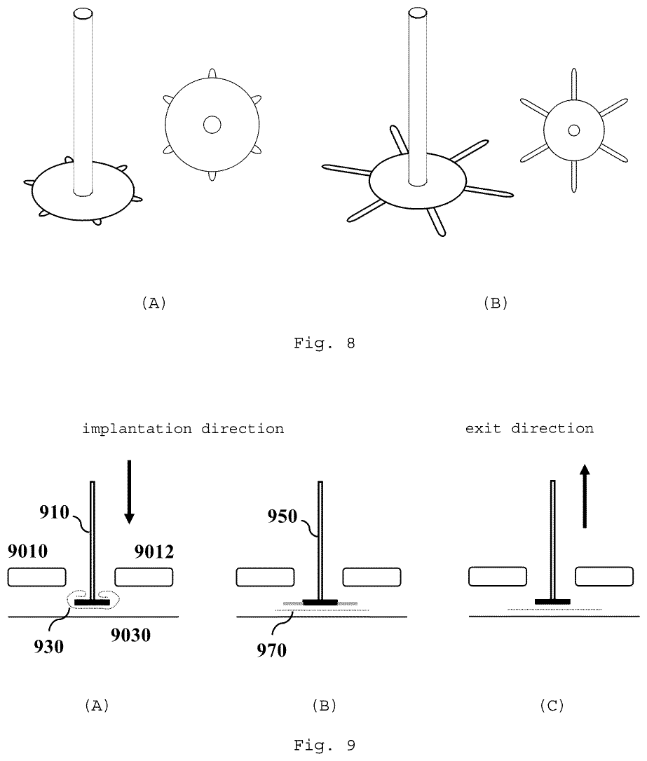

FIGS. 8 to 9 show yet another non-limiting embodiment of a support bracket for an ECOG electrode. FIG. 8(A) shows a shape of a support bracket, where a hard handle and a flower-like support bracket body are included, the support bracket being shown in FIG. 8(B) when unfolded. In particular, the support bracket may be unfolded by a mechanical device such as pneumatic pressure, a linkage, or other implementations. FIG. 9 is a schematic view of a step of implanting an ECOG electrode via a support bracket according to the foregoing embodiment. As shown in FIG. 9(A), a support bracket 910, which has thereon carried an ECOG electrode 930 wrapped on the support bracket 910, is implanted, from a gap between skulls 9010 and 9012, between the skulls and a cerebrum surface 9030. The support bracket 910 delivers the electrode 930 into a designated position in a rolled state in an implantation direction indicated by an arrow in FIG. 9(A), and then, as shown in FIG. 9(B), unfolds the support bracket 910 into a support bracket 950 in a form as shown in FIG. 8(B), such that the ECOG electrode 920 is, between the skull 9010/9012 and the cerebrum surface 9030, flattened in a form of an electrode 970. Then, the support bracket 910 is changed to its original folded state and withdrawn in an exit direction indicated by an arrow in FIG. 9(C), thereby completing the flattening and implantation of the electrode.

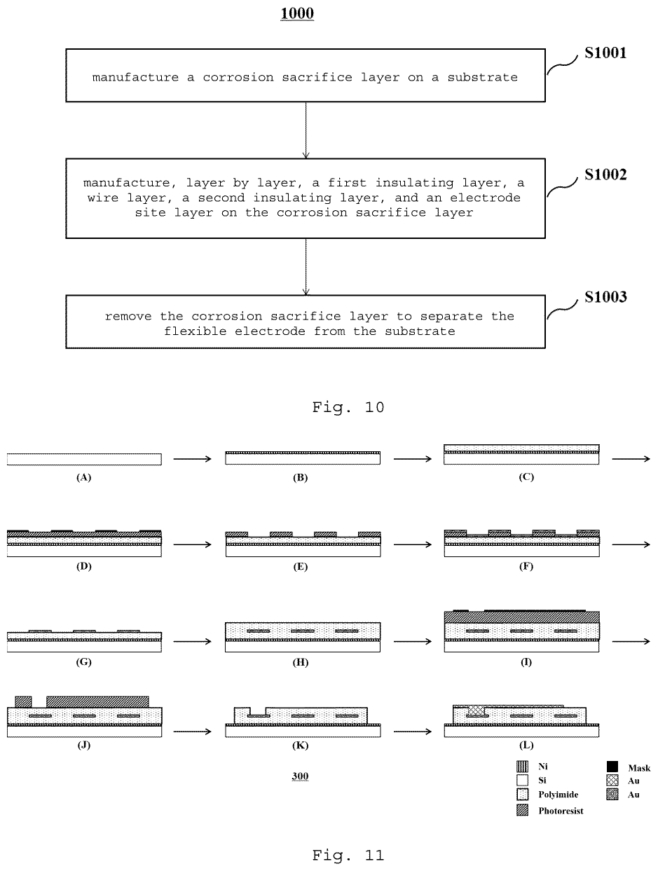

FIG. 10 is a flow diagram n illustrating a method for manufacturing a flexible electrode according to an embodiment of the present disclosure. In the present disclosure, a flexible electrode in nanometers may be manufactured by using a manufacturing method based on a micro-electro mechanical system (MEMS) process. The method 1000 may comprise: at S1001, manufacturing a flexible separation layer on a substrate; at S1002, manufacturing, layer by layer, a first insulating layer, a wire layer, a second insulating layer, and an electrode site layer on the flexible separation layer, wherein before manufacturing the electrode site layer, a via hole is manufactured at a position in the first insulating layer that corresponds to an electrode site by a patterning method; and at S1003, removing the flexible separation layer to separate the flexible electrode from the substrate.

FIG. 11 is a schematic view illustrating a method for manufacturing a flexible electrode according to an embodiment of the present disclosure. The manufacturing processes and structures of the flexible separation layer, bottom insulating layer, wire layer, top insulating layer, electrode site layer, etc. of the flexible electrode will be described in more detail in conjunction with FIG. 11.

View (A) of FIG. 11 shows a substrate of the electrode. In the embodiment according to the present disclosure, a hard substrate such as glass, quartz, or silicon wafer may be adopted. In the embodiment of the present disclosure, another soft material may be adopted as the substrate, for example, a same material as the insulating layer.

View (B) of FIG. 11 shows a step of manufacturing a flexible separation layer on the substrate. The flexible separation layer may be removed by applying a specific substance, thereby facilitating separation of the flexible part of the electrode from the hard substrate. In the embodiment shown in FIG. 11, Ni is used as a material of the flexible separation layer, and another material, such as Cr and Al, may also be used. In the embodiment according to the present disclosure, when the flexible separation layer is manufactured on the substrate by evaporation, part of the exposed substrate may be etched first, thereby improving flatness of the entire substrate after the evaporation. It should be appreciated that the flexible separation layer is an optional rather than necessary part of the flexible electrode. According to properties of the selected material, the flexible electrode can also be easily separated without the flexible separation layer. In the embodiment according to the present disclosure, the flexible separation layer may also have a mark thereon, which may be used for alignment of a subsequent layer.

View (C) of FIG. 11 shows manufacturing a bottom insulating layer on the flexible separation layer. As a non-limiting example, when a polyimide material is taken for the insulating layer, the manufacturing of the bottom insulating layer may include steps of film formation processing, film formation curing, and reinforced curing to manufacture a film as the insulating layer. The film formation processing may include coating polyimide on the flexible separation layer, for example, a layer of polyimide may be spin coated at segmented speeds. The film formation curing may include step-wise temperature increase to a higher temperature and heat preservation for film formation, thereby performing subsequent processing steps. The reinforced curing may include multi-gradient temperature increase, preferably in the presence of a vacuum or nitrogen atmosphere, and several hours of baking, before the subsequent layers are manufactured. It should be understood that the above manufacturing process is merely a non-limiting example of the manufacturing process for the bottom insulating layer, and one or more steps thereof may be omitted, or more other steps may be included.

It should be noted that the above manufacturing process is aimed at an embodiment in which a bottom insulating layer in a flexible electrode without a bottom electrode site layer is manufactured and there is no via hole corresponding to an electrode site in the bottom insulating layer. If the flexible electrode includes a bottom electrode site layer, the bottom electrode site layer may be manufactured on the flexible separation layer before the bottom insulating layer is manufactured. For example, Au and Ti may be sequentially evaporated on the flexible separation layer. A patterning step of a bottom electrode site will be detailed in the following description of a top electrode site. Accordingly, when the flexible electrode includes the bottom electrode site, in the process of manufacturing the bottom insulating layer, in addition to the above steps, a patterning step may be further included, for etching a via hole at a position in the bottom insulating layer that corresponds to the bottom electrode site. The patterning step of the insulating layer will be detailed later in the following description of a top insulating layer.

Views (D) to (G) of FIG. 11 show manufacturing a wire layer on the bottom insulating layer. As shown in view (D), photoresist and mask may be applied on the bottom insulating layer. It should be understood that a patterned film may be prepared using other lithography means, such as s laser direct writing and e-beam lithography. By setting a pattern of the mask related to the wire layer, for example, the pattern of the wire layer 240 shown in FIG. 1, i.e., a contour of one or more wires of wire electrodes extending from a backend, can be implemented. Next, exposure and development may be performed to obtain a structure as shown in view (E). In the embodiment according to the present disclosure, for patterns of different sizes, different developers and concentrations thereof may be adopted. In this step, layer-to-layer alignment may also be included. Next, film formation may be performed on the structure shown in view (E), for example, a process such as evaporation and sputtering may be used to deposit a metal film material, such as Au, to obtain a structure shown in view (F). Next, lift-off may be performed to separate, by removing photoresist in a non-patterned area, a film in the non-patterned area from a film in a patterned area, to obtain a structure as shown in view (G), i.e., the wire layer manufactured.

In the embodiment according to the present disclosure, a backend site layer may also be manufactured before the wire layer is manufactured. As a non-limiting example, a manufacturing process of the backend site layer may be similar to that of the metal film in the foregoing description of the wire layer.

Views (H) to (K) of FIG. 11 show manufacturing a top insulating layer. For a photosensitive film, generally, patterning can be implemented directly by patterning exposure and development, but for a non-photosensitive material used for the insulating layer, patterning cannot be implemented by exposing and developing the material itself, so that possible to manufacture, on this layer, a sufficiently thick patterned anti-etching layer, then remove a film in an area not covered by the anti-etching layer by dry etching, and then remove the anti-etching layer to pattern the non-photosensitive layer. As a non-limiting example, for the manufacturing of the insulating layer, photoresist may be taken as the anti-etching layer. The manufacturing of the top insulating layer may include steps of film formation processing, film formation curing, patterning, reinforced curing, etc., wherein view (H) shows a structure obtained after film formation of the top insulating layer, view (I) shows applying photoresist and a mask on the top insulating layer after the film formation, view (J) shows a structure including the anti-etching layer obtained after the exposure and development, and view (K) shows a structure including the manufactured top insulating layer. The film formation processing, film formation curing, and reinforced curing have been detailed in the foregoing description of the bottom insulating layer, and are omitted here for the sake of brevity. The patterning step can be performed after film formation curing, or after reinforced curing, and the anti-etching capability of the insulating layer after the reinforced curing is stronger. Specifically, in the view (I), a layer of sufficiently thick photoresist is manufactured on the insulating layer by steps of spin-on PR coating, baking and the like. By setting a pattern of the mask related to the top insulating layer, for example, the pattern of the first insulating layer shown in FIG. 1 can be implemented, i.e., a contour of the top insulating layer implemented on one or more wires in wire electrodes extending from the backend and a contour of a via hole implemented at a position in the top insulating layer that corresponds to an electrode site. In the view (J), the pattern is transferred onto the photoresist on the insulating layer by steps such as exposure and development to obtain the anti-etching layer, wherein, a part to be removed from the top insulating layer is exposed. It is possible to remove the exposed part of the top insulating layer by dry etching, and after flood exposure, remove remaining photoresist on the top insulating layer by using a developer or acetone, etc., to obtain a structure shown in view (K).

In the embodiment according to the present disclosure, before manufacturing the top insulating layer, it may be further tackified, to improve a bonding force between the bottom insulating layer and the top insulating layer.

View (L) of FIG. 11 also shows manufacturing a top electrode site layer on the top insulating layer.

In the manufacturing process of the ECOG electrode of the present application, the electrode can be customized in different sizes, shapes, sites, and distributions according to a specific cerebrum modeled by MRI/CT three-dimensional reconstruction. This facilitates that the ECOG electrode is applicable to different scenes, different cerebrum areas, different objects, strengthening the flexibility of the electrode. Since the electrode can be compatible with the MRI/CT and does not affect MRI/CT radiography, the electrode and the MRI/CT can be synchronously tested. In addition, a MRI/CT developer can be added when needed for determining an implantation position of the electrode in the cerebrum.

A backend interface of the electrode can be designed and customized according to actual requirements so as to be integrated with a coupling signal preprocessing system, such as using a wire connection or a cable connection, and has good compatibility with different signal acquisition devices. The electrode can integrate part of a signal preprocessing circuit or chip with the ECOG electrode by a micro-nano processing technology for co-implantation into the cerebral cortex or under the cerebral cortex, so that a front-end integrated system where the chip and the ECOG electrode are integrated is implemented, and while the device volume is reduced, by integrating a signal wireless transmission mode including and not limited to Bluetooth, serial port and the like, signal acquisition is not limited in a simulation environment of a laboratory any more, which is of important significance for researching cerebral cortex signal activities of a subject in a natural environment.

The terms “front”, “back”, “top”, “bottom”, “above”, “below”, and the like in the description and claims, if any, are used for descriptive purposes and not necessarily for describing permanent relative positions. It should be understood that the terms so used are interchangeable where appropriate, such that the embodiments of the present disclosure described herein are, for example, capable of operation in other orientations different from those shown herein or otherwise described.

As used herein, the term “exemplary” means “serving as an example, instance, or illustration”, and not as a “model” that is to be reproduced exactly. Any implementation exemplarily described herein is not necessarily to be construed as preferred or advantageous over other implementations. Moreover, the present disclosure is not limited by any expressed or implied theory presented in the above TECHNICAL FIELD, BACKGROUND, SUMMARY, or

### DETAILED DESCRIPTION

As used herein, the term “substantially” means encompassing any minor variations caused by imperfections in design or manufacturing, tolerances of devices or components, environmental effects and/or other factors. The term “substantially” also allows for differences from a perfect or ideal situation caused by parasitic effect, noise, and other practical considerations that may exist in a practical implementation.

For reference purposes only, similar terms such as “first” and “second” can be used herein, and thus are not intended to be limiting. For example, unless clearly indicated by the context, the terms “first”, “second” and other such numerical terms involving structures or elements do not imply a sequence or order.

It should be further understood that the term “comprise/include”, when used herein, specifies the presence of stated features, entireties, steps, operations, units, and/or components, but do not preclude the presence or addition of one or more other features, entireties, steps, operations, units, components, and/or combinations thereof.

As used herein, the terms “and/or” includes any and all combinations of one or more of listed items in association. Terms used herein are for the purpose of describing specific embodiments only and are not intended to limit the present disclosure. As used herein, singular forms “a”, “an”, and “the” are also intended to include plural forms, unless clearly indicated by the context otherwise.

Those skilled in the art should realize that boundaries between the above operations are merely illustrative. Multiple operations can be combined into a single operation, the single operation can be distributed in additional operations, and the execution of the operations can be at least partially overlapped in time. Moreover, an alternative embodiment can include multiple instances of specific operations, and the order of the operations may be altered in various other embodiments. However, other modifications, variations, and alternatives are also possible.

Accordingly, the description and the accompanying drawings should be regarded as illustrative rather than restrictive.

Although some specific embodiments of the present disclosure have been described in detail by means of examples, it should be understood by those skilled in the art that the above examples are for illustration only and are not intended to limit the scope of the present disclosure. The embodiments disclosed herein can be combined arbitrarily without departing from the spirit and scope of the present disclosure. Those skilled in the art should also appreciate that various modifications can be made to the embodiments without departing from the scope and spirit of the present disclosure. The scope of the present disclosure is defined by the appended claims.

1. A surface electrode for a central nervous system, comprising:
at least one implantable and flexible electrode sheet, wherein each of the at least one electrode sheet comprises:
a wire, located between a first insulating layer and a second insulating layer of the flexible electrode; and
an electrode site, located on an outer surface of at least one insulating layer of the first insulating layer or the second insulating layer and electrically coupled to the wire through a via hole in the at least one insulating layer,
wherein the surface electrode is configured to be flattened to fit against a biological tissue surface of the central nervous system after implantation.

2. The surface electrode according to claim 1, wherein
the surface electrode is configured to be implanted to the biological tissue surface of the central nervous system using a support bracket.

3. The surface electrode according to claim 2, wherein
the support bracket has a micromechanical mechanism, comprising a cantilever beam, a latch, or a linkage mechanism.

4. The surface electrode according to claim 2, wherein
the support bracket is configured to implant the surface electrode by microfluidics.

5. The surface electrode according to claim 2, wherein
a material of the support bracket comprises any one or a combination of metal and alloy such as tungsten, platinum, titanium, and magnesium, a polymer material such as polyimide, polydimethylsiloxane (PDMS), hydrogel, epoxy, and polyethylene, and chitosan and polyethylene glycol (PEG).

6. The surface electrode according to claim 2, wherein
the support bracket is configured to implant the at least one electrode sheet into a craniotomy site with significant trauma, the part comprising a frontal lobe, occipital lobe, temporal lobe, or central cerebral great vessel.

7. The surface electrode according to claim 2, wherein
the support bracket is configured to implant the at least one electrode sheet along a gap between a central nervous tissue and a bone.

8. The surface electrode according to claim 1, wherein
the surface electrode is configured to be in a flattened, rolled, or wrapped state during the implantation and removed from a cerebrum in the flattened, rolled, or wrapped state.

9. The surface electrode according to claim 1, wherein
the wire in each electrode sheet comprises a plurality of wires located in a wire layer of the flexible electrode and spaced apart from each other, and
electrode sites in each electrode sheet comprises a plurality of electrode sites each electrically coupled with one of the plurality of wires through corresponding via holes in the second insulating layer.

10. The surface electrode according to claim 9, wherein
the wire has a width of 10 nm to 500 μm.

11. The surface electrode according to claim 1, wherein
a backend portion comprises at least one backend site,
wherein, the at least one electrode sheet each extends to the backend portion, and
each backend site is electrically coupled to one of wires and a backend circuit through a via hole in the first insulating layer or the second insulating layer, to implement bidirectional signal transmission between the electrode site electrically coupled to the one of the wires and the backend circuit.

12. The surface electrode according to claim 1, wherein
a material of the first insulating layer and the second insulating layer is any one of polyimide, polydimethylsiloxane, parylene, epoxy, polyamide imide, polylactic acid, polylactic acid hydroxyacetic acid copolymer, SU-8 photoresist, silica gel, and silicone rubber, or a combination thereof.

13. The surface electrode according to claim 11, wherein
the first insulating layer and the second insulating layer have a thickness of 100 nm to 300 μm.

14. The surface electrode according to claim 1, wherein
the electrode site and the wire in each electrode sheet comprise a conductive layer and an adhesion layer, respectively.

15. The surface electrode according to claim 14, wherein
a material of the conductive layer is any one gold, platinum, iridium, tungsten, magnesium, molybdenum, platinum-iridium alloy, titanium alloy, graphite, and carbon nanotube or a combination thereof, and the conductive layer has a thickness of 5 nm to 2 μm, and
a material of the adhesion layer comprises titanium (Ti), titanium nitride (TiN), chromium (Cr), tantalum (Ta), tantalum nitride (TaN), and the adhesion layer has a thickness of 1 to 50 nm.

16. The surface electrode according to claim 15, wherein
when the material of the conductive layer is metal, the conductive layer further comprises a surface treatment layer, a material of the surface treatment layer being any one of or a combination of PEDOT, iridium dioxide, porous gold, and platinum black (Pt black).

17. The surface electrode according to claim 1, further comprising:
a flexible separation layer, a material of the flexible separation layer comprising nickel (Ni), chromium (Cr), or aluminum (AI), and the flexible separation layer being configured to be removed by a specific substance to avoid affecting the at least one electrode sheet.

18. The surface electrode according to claim 1, wherein the surface electrode is configured to be customized according to a specific cerebrum shape modeled by three-dimensional reconstruction obtained by medical imaging means (MRI/CT).

19. The surface electrode according to claim 1, wherein the surface electrode is configured to be compatible with the MRI/CT.

20. A method for manufacturing a surface electrode for a central nervous system, the surface electrode comprising the surface electrode according to claim 1, the method comprising:
manufacturing, layer by layer, the first insulating layer, a wire layer, the second insulating layer and an electrode site layer,
wherein, before manufacturing the electrode site layer, the via hole is manufactured at a position in the second insulating layer that corresponds to the electrode site by a patterning method.

21. The method for manufacturing according to claim 20, further comprising:
manufacturing a flexible separation layer on a substrate;
manufacturing the first insulating layer, the wire layer, the second insulating layer and the electrode site layer on the flexible separation layer; and
removing the flexible separation layer to separate the flexible electrode from the substrate.

22. The method for manufacturing according to claim 20, wherein
a shape, size, and site distribution of the surface electrode are determined according to a specific cerebrum modeled by three-dimensional reconstruction obtained by medical imaging means (MRI/CT).
